(19) United States
(12) Patent Application Publication
(10) Pub. No.: US 2026/0022093 A1
(43) Pub. Date: Jan. 22, 2026

(54) MANUFACTURE OF XANTHATE BY CONTINUOUS CONVERSION

(71) Applicant: Cary Rider EDWARDS, Scottsdale, AZ (US)

(72) Inventor: Cary Rider EDWARDS, Scottsdale, AZ (US)

(21) Appl. No.: 19/272,360

(22) Filed: Jul. 17, 2025

(30) Foreign Application Priority Data
Jul. 18, 2024 (ZA) 2024/05582

(51) Int. Cl. C07C 329/14

(52) U.S. Cl. CPC C07C 329/14

(57) ABSTRACT

A method for the manufacture of high purity xanthate continuously in a system of reactors in a liquid organic medium by reacting caustic and alcohol to form alcoholate in a large circulating charge of previously reacted alcoholate at an elevated temperature, preferably no more than 110° C. A portion of the alcoholate is continuously withdrawn, cooled preferably to no more than 35° C. and rapidly dispersed with carbon disulfide to form xanthate in a large circulating charge of previously reacted xanthate while maintaining precise temperature control. Average contact time with byproduct water is 10 minutes. The product is continuously withdrawn and immediately dried to obtain in excess of 95% pure product.

### BACKGROUND OF THE INVENTION

The present invention relates to the preparation of xanthate, a chemical compound of known utility for the processing of minerals.

Xanthate is a chemical compound formed by the reaction of an alkali metal alcoholate and carbon disulfide. The alcoholate is formed by the reaction of an alcohol, commonly alcohols with between 2 and 8 carbon atoms, and an alkali metal, commonly sodium or potassium hydroxide.

### Alcoholate Reaction

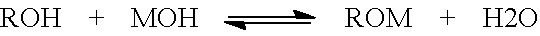

(Where R is an alkyl group and M is an alkali metal)

### Xanthate Reaction

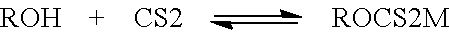

(Where R is an alkyl group and M is an alkali metal)

Xanthate has been used as a reagent in the sulfide flotation of minerals since discovery by Keller in his 1925 patent.

Xanthate is commonly commercially produced by two processes, the solid process and the liquid process.

In the solid process, alcohol is reacted with caustic, commonly anhydrous, then reacted with carbon disulfide to form xanthate. The reactants in the solid process are commonly used in approximately 1 to 1 to 1 mole ratios. The solid process has the advantages of requires no or minimal drying equipment, requires no solvent storage and recovery equipment, has lower plant investment cost as compared to the liquid process. The solid process has the disadvantages of the mixing equipment requires high power motors per unit of mixer volume necessitating smaller mixer volumes, reduced heat transfer rates between the reactants and the mixer shell or heat exchanger lead to reduced reactant feed rates as compared to the liquid process, the localized temperatures at the reactant introduction site are generally higher as compared to the liquid process and lead to reduced yields and lower purities. The solid process has an additional disadvantage of requiring expensive low temperature glycol or brine chillers to provide the cooling because of the temperature requirements and low heat transfer rates.

In the liquid process, a liquid organic medium, of which many are suitable (reference McCool, U.S. Pat. No. 2,678,939, 1954), is added to a reactor along with the reactant alcohol. The caustic is then added as either an aqueous solution or anhydrous and reacted to form the alcoholate and byproduct water. Carbon disulfide is then added to the reactor to form xanthate. The product is then commonly transferred to a dryer to remove the liquid organic medium and byproduct water. The liquid process has the advantages of higher yields and purities as compared to the solid process. The liquid process has the disadvantages of requiring drying, recovery, and purification equipment and associated costs, also increased energy requirements associated with the drying and recovery process. The liquid process has an additional disadvantage of often requiring expensive chillers to maintain the reactor temperature requirements.

Impurities in xanthate including water from formation of the alcoholate, unreacted caustic, sulfides, carbonates, and trithiocarbonates are detrimental to the product and can contribute to accelerated decomposition and lack of effectiveness of the xanthate product in the mineral processing application. Sodium trithiocarbonate (CNa2S3) is an impurity in some xanthates produced by the reaction of carbon disulfide and sodium hydroxide. Sodium trithiocarbonate has been shown to be a depressant for some sulfide minerals (see U.S. Pat. No. 4,510,050). Water accelerates the decomposition of xanthate into alcohol, carbon disulfide, carbonates, and trithiocarbonates. Water is formed in the manufacture of xanthate and therefore must be very quickly removed in order to prevent impurity reactions and decomposition of the xanthate product. Commercial xanthate purities are commonly 90% and as low as 84%.

### SUMMARY OF THE INVENTION

A general object of the present invention is to provide an improved method of producing xanthate.

Another object of the present invention is the continuous production of xanthate.

Another object of the present invention is to provide a method of producing xanthate of greater than 95% purity.

Another object of the present invention is the reduction trithiocarbonate impurities.

Another object of the present invention is improved energy efficiency through steady state operation of the process.

Another object of the present invention is the minimization of the xanthate contact time with the byproduct water.

Another object of the present invention is the high dispersion rate of the caustic in the reactor.

Another object of the present invention is the preheating of the reactant alcohol and liquid organic medium.

Another object of the present invention is the mechanical wiping action in the alcoholate precooler.

According to the present invention, there is provided for a process for the manufacture of xanthate by continuous conversion in a liquid organic medium, wherein the process comprises steps of:
a. continuously adding an anhydrous alkali metal hydroxide and the liquid organic medium to a caustic feeder reactor;
b. continuously withdrawing said alkali metal hydroxide and liquid organic medium as a feed into an alcoholate reactor;
c. introducing said alkali metal hydroxide, liquid organic medium, and a separate alcohol reactant feed into a large recirculating stream of preformed alcoholate from the alcoholate reactor;
d. rapidly mixing and immediately cooling said combined alcoholate recirculating stream to control temperature;
e. continuously removing a portion of said alcoholate recirculating stream as alcoholate feed into a xanthate reactor;
f. introducing said alcoholate feed and a separate carbon disulfide reactant feed into a large recirculating stream of preformed xanthate from the xanthate reactor;
g. rapidly mixing and immediately cooling said combined xanthate recirculating stream to control temperature;
h. continuously removing a portion of said xanthate recirculating stream as xanthate product; and
i. immediately drying the xanthate product to remove and recover a byproduct water, the liquid organic medium, any excess carbon disulfide and alcohol, leaving xanthate product of high purity.

In a first embodiment of the process, the anhydrous alkali metal hydroxide may be continuously introduced at ambient pressure and at or close to ambient temperature.

In a second embodiment, the separation of the anhydrous alkali metal hydroxide caustic feed process step from the alcoholate reactor process step and into separate reactors can allow the caustic feeder reactor to be continuously operated at ambient pressure and temperature and the alcoholate reactor can be continuously operated above ambient pressure and temperature.

In another embodiment of the process, the process may comprise an additional step wherein the anhydrous alkali metal hydroxide can be continuously introduced through a dual nitrogen blanketed airlock eliminating the escape of fumes.

According to another embodiment, the process may comprise an additional step wherein the anhydrous alkali metal hydroxide is continuously finely ground prior to the introduction into the caustic feeder reactor.

In another embodiment of the process, the alkali metal hydroxide, alcohol, and liquid organic medium may be preheated to 70° C. prior to the reaction forming the alcoholate reduces localized cold zones in the alcoholate reaction and improves yield and final product purity.

According to a further embodiment, the alkali metal hydroxide caustic feed stream may comprise 10% of the alcoholate reactor recirculating stream and minimizes temperature increases caused by the reaction of caustic and alcohol when combined in the recirculating stream.

According to another embodiment of the process, the alcoholate reactor may be maintained at a temperature in the range of 70° C. to 110° C.; and the temperature of the alcoholate reactor can be precisely controlled by the heat exchanger in the alcoholate recirculating stream. Optionally, the temperature of the alcoholate can be maintained at a steady state.

In a further embodiment of the process, the heat exchanger on the alcoholate recirculating stream can be cooled by higher temperature cooling water; wherein said higher temperature cooling water may not require refrigeration, and wherein said higher temperature cooling water can be cooled by more energy efficient evaporative cooling.

The process of the present invention further relates to energy efficiency of the xanthate manufacturing process preferably improved by continuous operation of the process; steady state temperature control of the reactors; separating the alcoholate and xanthate reaction into two reactors; and cooling the alcoholate reactor with higher temperature and more energy efficient evaporatively cooled water.

The process of the present invention may comprise an additional step wherein the alcoholate feed into the xanthate reactor recirculating stream is precooled to about 30° C.

According to another embodiment of the process, the alcoholate may be cooled in a heat exchanger which has a mechanical wiping action reduces alcoholate crystallization, build up, and fouling in said heat exchanger.

Optionally, the alcoholate feed may comprise less than 7% of the xanthate reactor recirculating stream of preformed xanthate. Typically, the large circulating stream of preformed xanthate and heat exchanger may prevent unwanted temperature rise of the xanthate recirculating stream after the introduction of the alcoholate feed and the carbon disulfide feed. Preferably, the temperature of the recirculating stream after the introduction of the alcoholate and the carbon disulfide feeds rises to no more than 40° C. More preferably, the heat exchanger in the xanthate recirculating stream can reduce the temperature of the recirculating stream to about 35° C. or less.

In another embodiment of the process, the temperature of the xanthate reactor is maintained at a steady state. The average holding time in the xanthate reactor, wherein the xanthate product is in contact with the byproduct water, may be about 10 minutes. Typically, the short contact time with the byproduct water may reduce xanthate product decomposition and impurities such that the final xanthate product purity of 95% or greater can be obtained.

According to another embodiment of the process, aqueous alkali metal hydroxide of 50 to 73% concentration by weight may be used instead of anhydrous alkali metal hydroxide and may be introduced into the alcoholate reactor recirculating stream without a significant effect on xanthate product purity after drying.

### BRIEF DESCRIPTION OF DRAWING

FIG. 1 represents a system view of some of the embodiments of the present invention and is presented in order to explain the present invention more clearly to those of ordinary skill in the art.

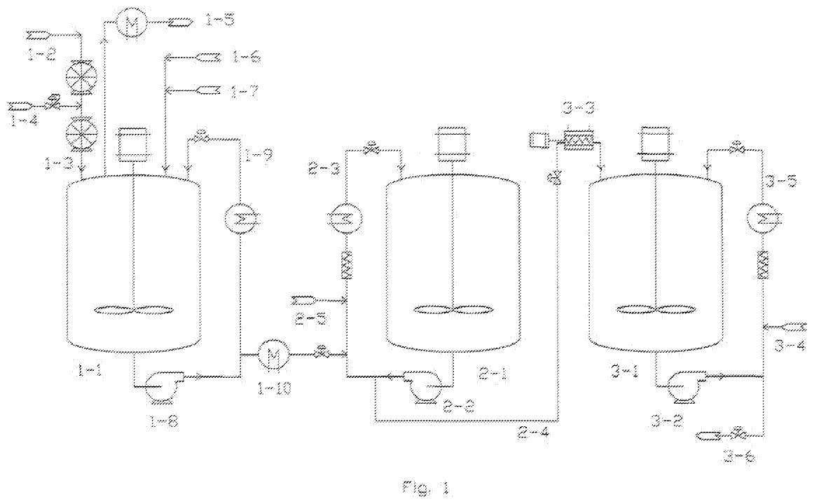

Reactor 1:1-1 Caustic Feeder Reactor; 1-2 Anhydrous Caustic Feed; 1-3 Dual Airlock; 1-4 Nitrogen Inert Gas Buffer; 1-5 Reactor Vent to Pollution Control System; 1-6 Solvent Feed; 1-7 Alcohol Feed; 1-8 Circulation Pump; 1-9 Recirculation Stream; 1-10 Caustic Feed to Alcoholate Reactor.

Reactor 2:2-1 Alcoholate Reactor; 2-2 Circulation Pump; 2-3 Recirculation Stream; 2-4 Alcoholate Feed to Xanthate Reactor; 2-5 Alternate Aqueous Caustic Feed.

Reactor 3:3-1 Xanthate Reactor; 3-2 Circulation Pump; 3-3 Alcoholate Precooler; 3-4 Carbon Disulfide Feed; 3-5 Recirculation Stream; 3-6 Xanthate Product Stream to Drying System.

### DETAILED DESCRIPTION OF THE INVENTION

The method of manufacture of xanthate in the present invention consists of three reactors, pumps, heat exchangers, mixers, and reactant feeders as described in the following detailed description. The reactant feeds are anhydrous or aqueous caustic, typically sodium hydroxide or potassium hydroxide, with the aqueous form being at a concentration of typically between 50% and 73%, an alcohol, and carbon disulfide.

In the manufacturing method of the present invention, provision has been made to utilize either anhydrous alkali metal hydroxide or in an alternate embodiment an aqueous solution of alkali metal hydroxide (both referred to herein as caustic). The purpose of the first reactor, the caustic feeder reactor, (reactor 1) is to provide a means to introduce finely ground anhydrous caustic into a liquid organic medium and separate the alcoholate reaction from the anhydrous caustic feed, There exist many suitable choices of liquid organic medium. The liquid organic medium is introduced continuously into reactor 1 in purified form without water or other impurities. The finely ground anhydrous caustic is continuously introduced into the reactor through a dual airlock mechanism. A small positive flow of nitrogen is introduced between the dual airlocks to prevent escape of fumes. Reactor 1 is cooled to a temperature to maintain a low vapor pressure so as to prevent escape of fumes. Reactor 1 is vented through a chilled condenser to a scrubber or other suitable air quality control system to prevent escape of fumes. Reactor 1 is agitated to maintain a uniform dispersion of anhydrous caustic in the liquid organic medium. A recirculation pump on reactor 1 continuously returns the major flow through a heat exchanger back to reactor 1, said heat exchanger maintains reactor 1 at the desired temperature during steady state operation and preferably below 40° C., the minor flow is taken continuously and is referred to herein as the caustic feed into the second reactor herein referred to as the alcoholate reactor and reactor 2. The average holding time in reactor 1 is short and preferably 10 to 15 minutes.

Reactor 2 is agitated to maintain a uniform distribution and contains preformed alcoholate, byproduct water, and the liquid organic medium. A large recirculating stream is pumped from reactor 2. The minor portion of said large recirculating stream is taken continuously and referred to as the alcoholate feed into reactor 3. The caustic feed coming from reactor 1 is preheated in a heat exchanger above 70° C. and introduced into the major portion of said large recirculating stream from reactor 2. The caustic feed comprising preferably 10% of the major portion of said recirculating stream, said 10% ratio allows temperature control of the heat of reaction of the combined recirculating stream. The reactant alcohol feed is preheated to 70° C. and added continuously to the major portion of the recirculating stream of reactor 2. Preheating of the caustic and alcohol reactants and liquid organic medium prior to introduction into the recirculating stream is important as it reduces local cold zones in the recirculating stream and hence agglomeration of the caustic thereby increasing xanthate yield and product purity. The combined caustic feed, alcohol and the recirculating stream is passed through a static mixer, then passes through a heat exchanger, and then returns to reactor 2. Said heat exchanger maintains reactor 2 during steady state operation above 70° C. and preferably less than 110° C. The high steady state operation temperature of reactor 2 allows less expensive and more energy efficient evaporatively cooled water to be used as the cooling medium, significantly reducing plant operating and capital expense. The average holding time in reactor 2 is preferably 20 to 30 minutes to ensure complete conversion of the caustic reactant.

Reactor 3 is agitated to maintain a uniform distribution and contains performed xanthate, byproduct water and the liquid organic medium. A large recirculating stream is pumped from reactor 3. The minor portion of said large recirculating stream is continuously taken as the product stream. The reactant carbon disulfide feed, preferably at a temperature of no more than 35° C. and precooled if required, is added to the major portion of the recirculating stream of reactor 3. The alcoholate feed from reactor 2 is precooled in a heat exchanger to preferably 30° C. to 35° C. The alcoholate feed is then introduced into the major portion of the recirculating stream of reactor 3. The combined alcoholate and carbon disulfide feeds comprising less than 10% of the recirculating stream and preferably less than 7% of the recirculating stream in order to maintain adequate temperature control during the reaction. The combined recirculating stream from reactor 3 is then passed through a static mixer and through a heat exchanger to cool to preferably no more than 35° C. and then returned to reactor 3. Temperatures in excess of 40° C. in the static mixer accelerate the formation of trithiocarbonates and should be avoided in plant operation by increasing cooling, reducing feed rates, or increasing recirculation rates. The average holding time in reactor 3 is preferably 10 minutes to ensure complete conversion of the alcoholate, this is the average contact time of the xanthate and water byproduct. For high purity xanthate, it is important that the contact time be kept to a minimum, as xanthate decomposes and produces impurities when in contact with water.

The continuous product stream containing the xanthate product, byproduct water, liquid organic medium, and any excess reactants is immediately dried to remove and recover the water, liquid organic medium, excess carbon disulfide and alcohol leaving 95% or greater pure xanthate product. Many types of commercial drying equipment exist and are suitable for this process.

In an alternate embodiment of the current invention an aqueous caustic solution is used instead of the anhydrous caustic solution. The aqueous caustic solution typically being of commercial grade and 50 to 73% caustic by weight, the remainder being water. In plant operation it is likely that all grades of caustic both aqueous and anhydrous will be used to make specific types and grades of xanthate. In this alternate embodiment reactor 1 is not used, the liquid organic medium is preheated above 70° C. and added to the major portion of the recirculating stream of reactor 2, the aqueous caustic is preheated above 70° C. and added to the major portion of recirculating stream. The remainder of reactor 2 and reactor 3 functions as described above.

1. A process for the manufacture of xanthate by continuous conversion in a liquid organic medium, wherein the process comprises steps of:
a. continuously adding an anhydrous alkali metal hydroxide and the liquid organic medium to a caustic feeder reactor;
b. continuously withdrawing said alkali metal hydroxide and liquid organic medium as a feed into an alcoholate reactor;
c. introducing said alkali metal hydroxide, liquid organic medium, and a separate alcohol reactant feed into a large recirculating stream of preformed alcoholate from the alcoholate reactor;
d. rapidly mixing and immediately cooling said combined alcoholate recirculating stream to control temperature;
e. continuously removing a portion of said alcoholate recirculating stream as alcoholate feed into a xanthate reactor;
f. introducing said alcoholate feed and a separate carbon disulfide reactant feed into a large recirculating stream of preformed xanthate from the xanthate reactor;
g. rapidly mixing and immediately cooling said combined xanthate recirculating stream to control temperature;
h. continuously removing a portion of said xanthate recirculating stream as xanthate product; and
i. immediately drying the xanthate product to remove and recover a byproduct water, the liquid organic medium, any excess carbon disulfide and alcohol, leaving xanthate product of high purity.

2. The process as claimed in claim 1, wherein the anhydrous alkali metal hydroxide is continuously introduced at ambient pressure and at or close to ambient temperature.

3. The process as claimed in claim 1, wherein the separation of the anhydrous alkali metal hydroxide caustic feed process step from the alcoholate reactor process step and into separate reactors allow the caustic feeder reactor to be continuously operated at ambient pressure and temperature and the alcoholate reactor to be continuously operated above ambient pressure and temperature.

4. The process as claimed in claim 1 comprising an additional step wherein the anhydrous alkali metal hydroxide is continuously introduced through a dual nitrogen blanketed airlock eliminating the escape of fumes.

5. The process as claimed in claim 1 comprising an additional step wherein the anhydrous alkali metal hydroxide is continuously finely ground prior to the introduction into the caustic feeder reactor.

6. The process as claimed in claim 1, wherein the alkali metal hydroxide, alcohol, and liquid organic medium are preheated to 70° C. prior to the reaction forming the alcoholate reduces localized cold zones in the alcoholate reaction and improves yield and final product purity.

7. The process as claimed in claim 1, wherein the alkali metal hydroxide caustic feed stream comprises 10% of the alcoholate reactor recirculating stream and minimizes temperature increases caused by the reaction of caustic and alcohol when combined in the recirculating stream.

8. The process as claimed in claim 1, wherein:
a. the alcoholate reactor is maintained at a temperature in the range of 70° C. to 110° C.; and
b. the temperature of the alcoholate reactor is precisely controlled by the heat exchanger in the alcoholate recirculating stream.

9. The process as claimed in claim 8, wherein the temperature of the alcoholate is maintained at a steady state.

10. The process as claimed in claim 8, wherein:
a. the heat exchanger on the alcoholate recirculating stream is cooled by higher temperature cooling water,
b, wherein said higher temperature cooling water does not require refrigeration, and
c, wherein said higher temperature cooling water could be cooled by more energy efficient evaporative cooling.

11. The process as claimed in claim 1, wherein the energy efficiency of the xanthate manufacturing process is improved by:
a. continuous operation of the process;
b. steady state temperature control of the reactors;
c. separating the alcoholate and xanthate reaction into two reactors; and
d. cooling the alcoholate reactor with higher temperature and more energy efficient evaporatively cooled water.

12. The process as claimed in claim 1 comprising an additional step wherein the alcoholate feed into the xanthate reactor recirculating stream is precooled to about 30° C.

13. The process as claimed in claim 1, wherein the alcoholate is cooled in a heat exchanger which has a mechanical wiping action reduces alcoholate crystallization, build up, and fouling in said heat exchanger.

14. The process as claimed in claim 12, wherein the alcoholate feed comprises less than 7% of the xanthate reactor recirculating stream of preformed xanthate.

15. The process as claimed in claim 14, wherein the large circulating stream of preformed xanthate and heat exchanger prevent unwanted temperature rise of the xanthate recirculating stream after the introduction of the alcoholate feed and the carbon disulfide feed.

16. The process as claimed in claim 14, wherein the temperature of the recirculating stream after the introduction of the alcoholate and the carbon disulfide feeds rises to no more than 40° C.

17. The process as claimed in claim 14, wherein the heat exchanger in the xanthate recirculating stream reduces the temperature of the recirculating stream to about 35° C. or less.

18. The process as claimed in claim 1, wherein the temperature of the xanthate reactor is maintained at a steady state.

19. The process as claimed in claim 1, wherein the average holding time in the xanthate reactor, wherein the xanthate product is in contact with the byproduct water, is 10 minutes.

20. The process as claimed in claim 19, wherein the short contact time with the byproduct water reduces xanthate product decomposition and impurities and the final xanthate product purity of 95% or greater is obtained.

21. The process as claimed in claim 1, wherein aqueous alkali metal hydroxide of 50 to 73% concentration by weight is used instead of anhydrous alkali metal hydroxide and introduced into the alcoholate reactor recirculating stream without a significant effect on xanthate product purity after drying.
